(19) United States
(12) Patent Application Publication
(10) Pub. No.: US 2022/0081583 A1
(43) Pub. Date: Mar. 17, 2022

(54) ULTRAVIOLET (UV) RADIATION-REFLECTIVE MATERIAL, SYSTEM, AND METHOD

(71) Applicant: Nevoa Life Sciences, Gilbert, AZ (US)

(72) Inventor: Emmett Manuel Cunningham, Richmond, VA (US)

(21) Appl. No.: 17/421,712

(22) PCT Filed: Jan. 15, 2020
(86) PCT No.: PCT/US2020/013641

§ 371 Date: Jul. 8, 2021

Related U.S. Application Data
(60) Provisional application No. 62/918,037, filed on Jan. 15, 2019.
(60) Provisional application No. 62/919,630, filed on Mar. 22, 2019.

(51) Int. Cl. C09D 127/18; A61L 9/20; A61L 2/10; C08K 3/38

(52) U.S. Cl. CPC C09D 127/18; A61L 9/20; A61L 2202/15; C08K 3/38; A61L 2202/11; A61L 2/10

(57) ABSTRACT

The present invention relates to disinfection, including the atmospheric disinfection of environments containing hard surfaces, and, more particularly, to the enhancement of the reflective surfaces to improve such disinfection, reduce the overall cost of disinfecting such surfaces, and the time required for such disinfection. According to one embodiment, the invention provides a composition comprising: a paint or coating material; and a reflective material having a reflectivity of at least about 90% and a coefficient of sliding friction of 0.10 or less.

### CROSS-REFERENCE TO RELATED APPLICATIONS

This application claims the benefit of co-pending US Provisional Patent Application Serial Nos. 62/918,037, filed 15 Jan. 2019 and 62/919,630, filed 22 Mar. 2019, each of which is incorporated herein as though fully set forth.

### BACKGROUND

The disinfection of hard surfaces, including ductwork, and the surrounding air mass in today's hospital and eldercare facilities is paramount to reducing hospital-acquired infections (HAIs), which are a major challenge to patient safety. In American hospitals alone, the Centers for Disease Control (CDC) estimates that HAIs account for an estimated 1.7 million infections and 99,000 associated deaths each year. This is not to imply that hospitals and eldercare facilities are the only areas requiring thorough disinfection or that would benefit from improved disinfection.

There are many different approaches to the problem, including:
Fogging hydrogen peroxide, which can be extremely dangerous, destroy or bleach products in the room, and take up to five hours.
Fogging chlorine-based products, which leave a residue, can destroy or bleach products in the room, and take up to two hours.
Fogging ozone, which can be extremely dangerous and hazardous to all mammals and requires protective equipment to operate.

### SUMMARY

The present invention relates to disinfection, including the atmospheric disinfection of environments containing hard surfaces, and, more particularly, to the enhancement of the reflective surfaces to improve such disinfection, and reduce the overall cost of such disinfection.

According to one embodiment, the invention provides a composition comprising: a paint material; and a reflective material having a UV reflectivity of at least about 90% and a coefficient of friction of 0.10 or less.

According to another embodiment, the invention provides an ultraviolet (UV) radiation system comprising: a UV lamp; and a reflective shield adjacent the UV lamp, the reflective shield including a reflective material having a UV reflectivity of at least about 90% and a coefficient of friction of 0.10 or less.

According to yet another embodiment, the invention provides a method of directing ultraviolet (UV) radiation, the method comprising: covering a surface with a composition including a reflective material having a UV reflectivity of at least about 90% and a coefficient of friction of 0.10 or less; and directing at least a portion of UV radiation emitted from a UV radiation source onto the covered surface, whereby UV radiation emitted from the UV radiation source is reflected from the covered surface.

According to still another embodiment, the invention provides an article of manufacture including a reflective material having a UV reflectivity of at least about 90% and a coefficient of friction of 0.10 or less.

### DETAILED DESCRIPTION

Ultraviolet (UV) light electromagnetic radiation disinfection systems are able to disinfect surfaces and areas according to the volume of UVC output and the reflectance of the environment in which the systems are employed. The reflectivity of the surface material on the walls of the room, ductwork, or furnishings greatly affects the performance of the device in achieving disinfection on the surfaces or in the air, particularly those that are out of the “line of sight” or in range of the UVC electromagnetic radiation wave, i.e., what are commonly referred to as “shadow areas.”

UV light, and in particular UVA and UVB electromagnetic radiation, are employed in grow lamps for the purpose of growing plants indoors. The reflectivity of the surface material on the walls, floors, ceilings, or furnishings of the room or space may greatly affect the performance of the device in achieving higher growth rates at lower cost and in less time. For example:
UVA and UVB can increase plant yields. According to a recent study, plants grow bigger and faster when UV light exposure is increased.
UVA and UVB can increase the level of nutrients from plants, improving nutritive value and taste.
UVC, which is also produced by grown lamps, make plants more resistant to fungal infections.
Recent studies revealed that UVA and UVB light have a great impact on plants growth pattern, chemistry, and transpiration processes.

Currently, normal paint products have a UV electromagnetic radiation refection rating of 20-40%. The reflectivity of the paint/coating and other related products can be greatly enhanced by the inclusion of material that has a very low static coefficient of friction and a very high UV electromagnetic radiation refection rating of 90-95%. Fluorocarbon resins are one such class of materials, with polytetrafluoroethylene (PTFE) being the most common example. TEFLON® is perhaps the best-known PTFE formulation and is available from The Chemours Company. PTFE has a coefficient of friction between 0.05 and 0.10. Expanded PTFE (eFTFE), such as that developed by W.L. Gore & Associates, Inc., is another such fluorocarbon resin.

Although fluorocarbon resins are likely the most commonly known materials with high-reflective properties, other materials may also be used according to the invention. For example, aluminum magnesium boride (Al₃Mg₃B₅₆, though often expressed nominally as AlMgB₁₄), commonly referred to as BAM, is a ceramic alloy that is highly resistive to wear and has a very low (0.02) coefficient of friction. BAM also has a UV reflectivity of 90-95%.

UVC disinfection of a typical room takes up to about one hour. The UVC machines come in many varieties, including robotic, stand-alone, pulsing, and flashing, some with mutable units. When installed inside ductwork, UV systems include tubes or light-emitting diodes (LEDs) that are permanently mounted and operate when the system is running.

However, as distance from a UV lamp increases, the effectiveness of UV radiation against microorganisms decreases sharply. This may require multiple positionings of the lamp around the area being disinfected in order to achieve even partial disinfection.

Wall surfaces and products painted with a PTFE/ePTFE-based paint or manufactured with the inclusion of a FTFE/eFTFE material can achieve up to 95% reflectivity, greatly enhancing the efficacy of UV disinfecting systems and reducing the number of times such systems or system components would need to be moved in order to achieve thorough disinfection.

Similarly, coating the surfaces of ductwork with PTFE/ePTFE-based paint or other coating materials would increase the reflectiveness of the surfaces from 30-60% (bare metal) to 90-95%. This can not only improve the disinfecting efficacy of UV disinfecting systems employed within the ductwork, but also reduce the number of tubes or LEDs required to achieve such disinfection.

Aspects of the present invention also relate to the introduction or increase of UV radiation to facilities in which plant products are grown, through the use of improved grow lamps. According to such aspects, the hard surfaces, including the reflective shields of the grow lamps, are coated or manufactured with a UV-reflective coating. This can reduce the number of grow lamps, the time that such grow lamps are on, and the overall cost of growing plants. This aspect is applicable to the production of any plant products, including, for example, tomatoes, lettuce, peppers, cucumbers, spinach, herbs (including marijuana), and strawberries.

Embodiments of the invention improve the ability of UV disinfection systems to focus the UV electromagnetic radiation onto all surfaces and likewise the atmosphere of the room, ductwork, or other environment being disinfected or in which plants are being grown. By adding a highly reflective material with a very low static coefficient of friction to the paints or coatings used to cover the walls, floors, or ceiling of the area, the reflectivity of the area is increased. In some cases, this increase in UV reflectivity can be up to 95%. In such areas, the UV electromagnetic radiation wave maintains its strength and overall germicidal or plant growth effectiveness. When PTFE is used to increase reflectivity, such maintenance of the UV wave is consistent at up to 80 degrees of deflection.

Increasing the reflectivity of hard surfaces can add other benefits as well, including:
Reduced fire hazard: the use of PTFE or ePTFE, according to the invention can greatly reduce the overall flammability of an area. PTFE has a melting point of 326.85° C. (620.33° F.; 600.00 K) and is not flammable, even as a liquid.
Prevention or reduction of leaked radiation: surfaces treated according to the invention reflect all radiation types, not just UV radiation.
Improved surface durability: materials employed according to embodiments of the invention have such low static coefficient of friction that they reduce the ability of foreign materials to stick to the treated surface.

According to embodiments of the invention, high-quality paints and coatings can be formulated to include PTFE and/or ePTFE, or other US-reflective additives, which provide the disinfection, durability, and other benefits noted above. Such formulations may include other additives as well, such as germicides and bactericides.

According to other embodiments of the invention, UV-reflective additives, such as PTFE, ePTFE, and/or BAM, may be incorporated into articles of manufacture during their production. Such embodiments may comprise virtually any material. Examples include, but are in no way limited to: floor tiles, countertops, ceramics, composite cabinetry and furniture components, household fabrics, and clothing.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A composition comprising:
a paint or coating material; and
a reflective material having a UV reflectivity of at least about 90% and a coefficient of friction of 0.10 or less.

2. The composition of claim 1, wherein the reflective material includes a fluorocarbon resin.

3. The composition of claim 2, wherein the fluorocarbon resin is polytetrafluoroethylene (PTFE).

4. The composition of claim 2, wherein the fluorocarbon resin is expanded polytetrafluoroethylene (ePTFE).

5. The composition of claim 1, wherein the reflective material includes aluminum magnesium boride (AlMgB₁₄).

6. An ultraviolet (UV) radiation system comprising:
a UV lamp; and
a reflective shield adjacent the UV lamp, the reflective shield including a reflective material having a UV reflectivity of at least about 90% and a coefficient of friction of 0.10 or less.

7. The UV radiation system of claim 6, wherein the reflective material includes a fluorocarbon resin.

8. The UV radiation system of claim 7, wherein the fluorocarbon resin is polytetrafluoroethylene (PTFE).

9. The UV radiation system of claim 7, wherein the fluorocarbon resin is expanded polytetrafluoroethylene (ePTFE).

10. The UV radiation system of claim 6, wherein the reflective material includes aluminum magnesium boride (AlMgB₁₄).

11. The UV radiation system of claim 6, wherein the UV lamp is capable of emitting any or all of UVA radiation, UVB radiation, and UVC radiation.

12. The UV radiation system of claim 6, wherein the reflective material is coated onto a surface of the reflective shield.

13. The UV radiation system of claim 6, wherein the reflective material is incorporated into a body of the reflective shield.

14. A method of directing ultraviolet (UV) radiation, the method comprising:
covering a surface with a composition including a reflective material having a UV reflectivity of at least about 90% and a coefficient of friction of 0.10 or less; and
directing at least a portion of UV radiation emitted from a UV radiation source onto the covered surface,
whereby UV radiation emitted from the UV radiation source is reflected from the covered surface.

15. The method of claim 14, wherein the reflective material includes a fluorocarbon resin selected from a group consisting of: polytetrafluoroethylene (PTFE) and expanded polytetrafluoroethylene (ePTFE).

16. (canceled)

17. (canceled)

18. The method of claim 14, wherein the reflective material includes aluminum magnesium boride (AlMgB₁₄).

19. The method of claim 14, wherein the UV radiation source is capable of emitting any or all of UVA radiation, UVB radiation, and UVC radiation.

20. The method of claim 14, wherein the surface is selected from a group consisting of: a wall, a floor, and a ceiling.

21. The method of claim 14, wherein the surface is an inner surface of a ductwork system.

22-27. (canceled)
